(19) United States
(12) Patent Application Publication
(10) Pub. No.: US 2024/0374508 A1
(43) Pub. Date: Nov. 14, 2024

(54) DEVICES AND METHODS OF USE THEREOF FOR NON-HORMONAL CONTRACEPTION

(71) Applicant: Elle, MD Biotechnologies Inc., New Minas (CA)

(72) Inventor: Jennifer Johnston, Port Williams (CA)

(21) Appl. No.: 18/661,570

(22) Filed: May 11, 2024

Related U.S. Application Data
(60) Provisional application No. 63/501,575, filed on May 11, 2023.

(51) Int. Cl. A61K 9/00; A61K 31/19; A61K 33/34; A61K 35/00; A61K 35/745; A61K 35/747; A61P 15/18

(52) U.S. Cl. CPC A61K 9/0036; A61K 31/19; A61K 33/34; A61K 35/745; A61K 35/747; A61P 15/18; A61K 2035/115

(57) ABSTRACT

Described herein are contraceptive devices comprising a torus-shaped elastomeric body and a bioactive agent coupled into the elastomeric body. The devices can be inserted into the vaginal region of a female reproductive system by a subject in need thereof for prophylactic contraception and/or emergency contraception.

### CROSS-REFERENCE TO RELATED APPLICATIONS

This application claims the benefit of U.S. Provisional Application 63/501,575 filed on May 11, 2023, the disclosure of which is incorporated herein by reference in its entirety.

### FIELD

The present disclosure is related to devices and methods for non-hormonal contraception and particularly, intravaginal contraceptive devices that include copper as a spermicidal or spermiostatic agent.

### BACKGROUND

Current contraceptive options use barrier devices, hormonal approaches, and spermicidal agents. Barrier devices such as condoms, contraceptive sponges, cervical caps and diaphragms often lack effectiveness due to errors in use and are generally less convenient. Although hormonal approaches such as oral contraceptive pills, patches, rings, injections, implants and intrauterine devices (IUDs) are potentially more reliable than barrier devices, they have been linked to an increased risk of breast cancer, cervical cancer, Crohn's disease, ulcerative colitis, lupus, multiple sclerosis, impaired glucose metabolism, reduced bone mineral density, reduced libido, headaches, breakthrough bleeding, depression, anxiety and suicide. Hormonal contraceptives using estrogen also increase the risk of deep vein thrombosis, pulmonary embolism, heart attack and stroke. This limits the number of women who are eligible and willing to use them. Spermicidal products including compositions like creams and jellies are again less reliable and can be unappealing or difficult to use. Both copper and hormonal IUDs require an invasive clinical procedure to be implanted and removed from the uterus. This can be painful and accessing a provider to perform the procedure can be difficult, for example, in developing countries or for those without adequate health insurance.

Copper IUDs can also cause a foreign body reaction and lead to heavier, or painful periods, a major deterrent for patients.

Therefore, there is a need for an effective, safe and user-friendly contraceptive option.

### SUMMARY

The present disclosure describes novel contraceptive devices configured to release an effective amount of at least one bioactive agent having spermicidal or spermiostatic properties into the vaginal region, the cervical region, and the endometrial region of a female reproductive system of a subject when positioned in the vaginal region of the female reproductive system. As set forth herein, the term “vaginal region” can refer to the positioning of the device described herein, which is intended to be positioned high in the vagina, thereby meaning that the “vaginal region” can refer to the region between the external OS of the cervix and the introitus of the vagina. To the extent a different meaning is ascribed to the vaginal region, the meaning would be immediately envisaged by a skilled artisan when viewing the term in conjunction with the surrounding context and specific claim terms.

A device described herein can be positioned in the vaginal region (i.e., a muscular canal approximately 5-13 cm long, which can vary by individual) of a female reproductive system. The walls of the vagina are lined with an outer, fibrous adventitia; a middle layer of smooth muscle; and an inner mucous membrane. The middle and inner layers allow the expansion of the vagina to accommodate the device. When the device is placed high in the vagina (i.e., proximal to the cervical opening), a bioactive agent is released which impairs sperm metabolic processes and motility, thereby inhibiting a sperm from fertilizing an egg. As set forth herein, the vaginal region can be defined as comprising the region between the external OS of the cervix and the introitus (opening of the vagina) and the cervical region includes internal OS, endocervix, ectocervix, and external OS. As the superior portion of the vagina meets the cervix (i.e., the narrow inferior portion of the uterus), a bioactive agent can be diffused throughout the vaginal region, from the vaginal region into the cervical region, from the cervical region into the endometrial region to prevent a sperm from reaching and/or fertilizing an egg.

The devices described herein have advantages over current contraceptive devices. First, the devices can be made without hormones and thus do not have any side effects associated with hormonal devices like Nuvaring®; second, devices described herein can be positioned in the vaginal region of a female reproductive system, not within the uterus and as such, it does not cause foreign body reaction and uterus tissue inflammation like copper IUDs; third, unlike copper IUDs, the devices described herein can be easily used without requiring clinical implantation; and fourth, the devices described herein can be used for emergency contraception within about 120 hours of unprotected intercourse by a subject in need thereof, which cannot be achieved by Nuvaring®. In addition, the devices described herein can provide unexpectedly improved outcomes by positioning the bioactive agent proximal to the cervix but within the vaginal region to allow for maximal interaction with the bioactive agent and the environment in the cervix and vaginal region and maximize the spermicidal or spermiostatic efficacy of the device compared to prior art devices. The devices described herein allow for unexpectedly improved contraception by maximizing the bioactive agent's concentrations in three areas (the vaginal region, the cervical region, and the uterus) to most effectively create spermicidal or spermiostatic results without placing a foreign object in an area (i.e., placing an IUD in the uterus) that causes adverse effects like pain and inflammation while also providing the benefits of a ring positioned in the vaginal region without the use of compounds like hormones. Consequently, the devices described herein are unexpectedly improved over prior art devices.

In certain embodiments, a contraceptive device described herein comprises a torus-shaped elastomeric body and at least one bioactive agent having spermicidal or spermiostatic properties. The elastomeric body can be permeable or impermeable. In certain embodiments, the at least one bioactive agent is present as one or more segments coupled to the elastomeric body. The external surface of the one or more segments can be aligned with the external surface of the elastomeric body. In certain embodiments, the at least one bioactive agent is present as plural inclusions, where the torus-shaped elastomeric body can be dotted with plural inclusions having the surfaces of the at least one bioactive agent exposed. In certain embodiments, plural inclusions are embedded within a torus-shaped elastomeric body where the surfaces of the at least one bioactive agent can be partially exposed, unexposed, or a combination thereof. In certain embodiments, the elastomeric body has one or more holes and a copper wire is placed inside the elastomeric body. In certain embodiments, the elastomeric body has one or more holes and apertures where the at least one bioactive agent can be stored as solids or solutions in the apertures. A bioactive agent can be in contact with the environment of the vaginal region through its exposed surfaces, a permeable membrane, or a permeable elastomeric body.

In certain embodiments, the at least one bioactive agent comprises copper-containing agents. In certain embodiment, the at least one bioactive is copper, a copper alloy, or a copper salt. In certain embodiments, the at least one bioactive is copper. In certain embodiments, a total exposed surface area of copper is about 50-1000 mm². In certain embodiments, the copper concentrations in the uterus can be about 3.0-20 pg/g or about 3.0-20.0 pg/ml (dry weight) (and ranges therebetween) from day 3 until the time of removal of the contraceptive device described herein (e.g. up to 1 month, up to 6 months, up to 1 year, up to 2 years, up to 3 years, up to 4 years, and up to about 5 years) following the insertion of a device described herein. In certain embodiments, the copper concentrations in the uterus are about 0.5-5.0 pg/g or about 0.5-5.0 pg/ml (wet weight) from day 3 to year 5 following the insertion of a device described herein. In certain embodiments, the copper concentration in the uterus increases for about at least one week following the insertion of a device described herein. In certain embodiments, a device described herein has a copper release for at least about one week. In certain embodiments, a device described herein can have a pearl index of about 0.8-5.0.

In certain embodiments, a contraceptive device described herein can be co-administered with one or more vaginal-health-promoting agents. In certain embodiments, the one or more vaginal-health-promoting agents are administered prior to, concurrent with, or after administration of a contraceptive device described here. In certain embodiments, the devices described here can be used for prophylactic contraception before intercourse. In certain embodiments, the devices described here can be used for emergency contraception within about 10 hours, 12 hours, 20 hours, 24 hours, 48 hours, 72 hours, or 120 hours of unprotected intercourse.

Also described herein are methods of making the contraceptive devices.

These and other features, aspects, and advantages of the present embodiments will become understood with reference to the following description, figures, and appended claims.

### BRIEF DESCRIPTION OF THE DRAWINGS

Reference will now be made, by way of example, to the accompanying drawings which show examples of embodiments of the present application, and in which:

FIG. 1A is an illustrative example of a female reproductive system in which methods and devices described herein can be applied;

FIG. 1B shows the regions where copper ions are concentrated when using devices described herein compared to some commercially-available devices;

FIG. 1C shows the positions in a female reproductive system using devices described herein compared to some commercially-available devices;

FIG. 2 shows an exemplary contraceptive device as an intravaginal ring, in accordance with embodiments of the present disclosure;

FIG. 3 shows an exemplary intravaginal ring embedded with a copper-containing agent, in accordance with embodiments of the present disclosure;

FIGS. 4A-4E show an exemplary intravaginal ring having at least one copper-containing segments, in accordance with embodiments of the present disclosure;

FIG. 5A shows a cross-sectional view of the exemplary intravaginal ring of FIG. 4A, in accordance with embodiments of the present disclosure;

FIG. 5B is a perspective view of an exemplary copper-containing segment, in accordance with embodiments of the present disclosure;

FIG. 5C is a cut-away view of an exemplary copper-containing segment, in accordance with embodiments of the present disclosure;

FIG. 6A shows a side view of the intravaginal ring of FIG. 4A, in accordance with embodiments of the present disclosure;

FIG. 6B shows a cross-sectional view of the intravaginal ring of FIG. 6A, in accordance with embodiments of the present disclosure;

FIG. 7A shows an exemplary embodiment of an intravaginal ring, in accordance with embodiments of the present disclosure;

FIG. 7B show exemplary embodiments of a molded thermoplastic skeleton of the intravaginal ring of FIG. 7A, in accordance with embodiments of the present disclosure;

FIG. 8 is a flowchart illustrating an exemplary method of using a contraceptive device, in accordance with embodiments of the present disclosure;

Similar reference numerals may have been used in different figures to denote similar components.

### DETAILED DESCRIPTION

Described herein are novel contraceptive devices that can be positioned in the vaginal region of a female reproductive system by a subject in need thereof and after being positioned, releases at least one bioactive agent having spermicidal or spermiostatic properties into the vaginal region, the cervical region, and the endometrial region for prophylactic contraception and/or emergency contraception. In certain embodiments, a contraceptive device described herein is an intravaginal ring comprising a torus-shaped elastomeric body and at least one bioactive agent coupled to the elastomeric body. In certain embodiments, the at least one bioactive agent is present as plural inclusions dotted or embedded on the elastomeric body. In certain embodiments, the at least one bioactive agent is present as one or more segments coupled into a torus-shaped elastomeric body. The external surface of the one or more segments can be aligned with the external surface of the elastomeric body. In certain embodiments, the elastomeric body has one or more holes and the at least one bioactive agent is placed within the elastomeric body. In certain embodiments, the elastomeric body has one or more holes and/or apertures where the at least one bioactive agent can be stored as solids or solutions in the apertures. In certain embodiments, a contraceptive device described herein further comprises a barrier.

In certain embodiments, an intravaginal ring described herein has a cross sectional diameter of about 3.0-10.0 mm and an external diameter of about 30-90 mm. In certain embodiments, an intravaginal ring described herein has a cross sectional diameter of about 4.0-10.0 mm and an external diameter of about 40-80 mm. In certain embodiments, an intravaginal ring described herein has a cross sectional diameter of about 4.0-9.0 mm and an external diameter of about 50-60 mm. In certain embodiments, an intravaginal ring described herein has a cross sectional diameter of about 5.5-9.0 mm and an external diameter of about 55-60 mm. In certain embodiments, an intravaginal ring described herein has a cross sectional diameter of about 5.7-6.1 mm or 6.3-7.5 mm and an external diameter of about 55-60 mm. In certain embodiments, an intravaginal ring described herein has a cross sectional diameter of about 6.35 mm and an external diameter of about 55 mm. In certain embodiments, an intravaginal ring described herein has a cross sectional diameter of about 5.6 mm and an external diameter of about 59.7 mm. In certain embodiments, an intravaginal ring described herein has a cross sectional diameter of about 6.2 mm and an external diameter of about 57.6 mm. In certain embodiments, an intravaginal ring described herein has a cross sectional diameter of about 7.9 mm and an external diameter of about 57.6 mm. In certain embodiments, an intravaginal ring described herein has a cross sectional diameter of about 9.0 mm and an external diameter of about 55 mm. In certain embodiments, an intravaginal ring described herein has a cross sectional diameter of about 4.0 mm and an external diameter of about 54 mm. In some embodiments, the inner diameter can be between about 25 mm and 70 mm, about 35 and 55 mm, about 40 and about 60 mm, about 25 and about 35 mm, about 35 and about 70 mm, and ranges therebetween. In some embodiments, the external diameter can be between about 35 mm and 80 mm, about 45 and 60 mm, about 50 and about 65 mm, about 25 and about 35 mm, about 50 and about 70 mm, and ranges therebetween.

In certain embodiments, an elastomeric body is made of a biocompatible polymer material, which can be permeable or impermeable. In certain embodiments, an elastomeric body is made of silicone. A silicone elastomer can be permeable or impermeable.

In certain embodiments, at least one bioactive agent is present as plural inclusions. In certain embodiments, an elastomeric body is dotted with plural inclusions (see e.g., FIG. 3) having the entire surface of the inclusions exposed. In certain embodiments, plural inclusions are embedded within an elastomeric body wherein the surface of the inclusions is partially exposed or is not exposed. In certain embodiments, plural inclusions have a total exposed surface area of about 50-1000 mm². In certain embodiments, the plural inclusions have a total exposed surface area of about 200-600 mm². In certain embodiments, the plural inclusions have a total exposed surface area of about 380-600 mm². In certain embodiments, the plural inclusions have a total exposed surface area of about 380-400 mm². In certain embodiments, the plural inclusions have a total exposed surface area of about 400-500 mm². In certain embodiments, the plural inclusions have a total exposed surface area of about 394 mm². In certain embodiments, the plural inclusions have a total exposed surface area of about 425 mm². In certain embodiments, the plural inclusions have a total exposed surface area of about 473 mm².

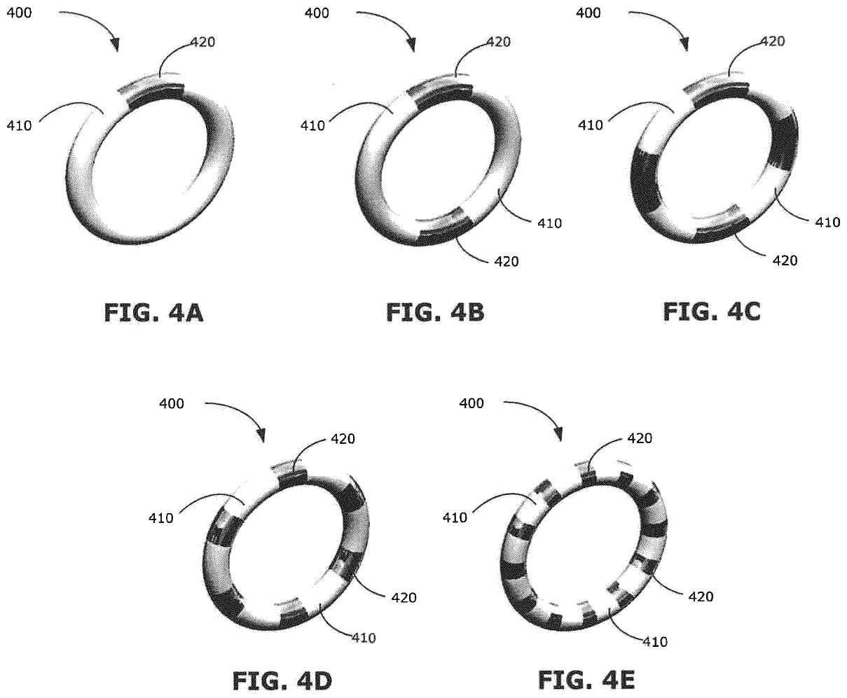

In certain embodiments, at least one bioactive agent is present as one or more segments that are coupled to an elastomeric body (see e.g., FIGS. 4A-4E). In certain embodiments, an elastomeric body further comprises a thermoplastic support ring. In certain embodiments, a biocompatible polymer material can be molded over a thermoplastic support ring to form an elastomeric body. The molding can be achieved by overmolding, inserting molding, or 3D printing. In certain embodiments, a thermoplastic support ring can be used to mount one or more bioactive agents-containing segments on it and thus connects the elastomeric body with the one or more segments. In certain embodiments, one or more bioactive agent-containing segments have a total exterior surface area of about 50-1000 mm². In certain embodiments, one or more bioactive agent-containing segments have a total exterior surface area of about 200-600 mm². In certain embodiments, one or more bioactive agent-containing segments have a total exterior surface area of about 380-600 mm². In certain embodiments, one or more bioactive agent-containing segments have a total exterior surface area of about 380-400 mm². In certain embodiments, one or more bioactive agent-containing segments have a total exterior surface area of about 400-500 mm². In certain embodiments, one or more bioactive agent-containing segments have a total exterior surface area of about 394 mm². In certain embodiments, one or more bioactive agent-containing segments have a total exterior surface area of about 425 mm². In certain embodiments, one or more bioactive agent-containing segments have a total exterior surface area of about 473 mm². In certain embodiments, one or more bioactive agent-containing segments has an arc of about 40-60 degrees. In certain embodiments, one or more bioactive agent-containing segments has an arc of about 45-50 degrees.

In certain embodiments, at least one bioactive agent is positioned in a permeable reservoir as a solution where the at least one bioactive agent is released via diffusion through the reservoir into a vaginal region. In certain embodiments, an elastomeric body has holes and apertures where the apertures can be used as reservoirs.

In certain embodiments, at least one bioactive agent is a copper-containing agent. Examples of a copper-containing agent include, but are not limited to, copper metal, copper alloys, copper salts, or any combinations thereof. Copper salts can be copper sulfate, copper gluconate, copper amino acid chelates, and copper oxide. In certain embodiments, at least one bioactive agent is copper metal. In certain embodiments, at least one bioactive agent may comprise one or more copper salts as solid. In certain embodiments, at least one bioactive agent may comprise one or more copper salts and one or more non-copper spermicide as solid. The solid can be embedded within an elastomeric ring and diffuse into the vaginal region, the cervical region, and the endometrial region when positioned in the vaginal region. In certain embodiments, at least one bioactive agent may comprise one or more copper salts in solution. In certain embodiments, at least one bioactive agent may comprise one or more copper salts and one or more non-copper spermicides in solution. The solution can be stored in a permeable reservoir that allows both copper salts non-copper spermicides to diffuse into the vaginal region when positioned therein. Examples of a non-copper spermicide include, but are not limited to nonoxynol-9, menfegol, boric acid, citric acid, zinc sulfate, potassium permanganate, and benzalknoim chloride.

In certain embodiments, at least one bioactive agent can further comprise one or more vaginal-health-promoting agents, which can be vaginal probiotics, prebiotics, acidifying agents, and a combination thereof. The vagina is home to a normal population of microorganisms (i.e., vaginal microbiota) that help to protect against infection by pathogenic bacteria, yeast, or other organisms that can enter the vagina. Administration of certain prebiotics and/or vaginal microbiota can promote the growth of microorganisms and thus are beneficial for promoting or maintaining a healthy vaginal environment. In addition, maintaining an acidic pH value (below 4.5) with acids can also protect the vagina from infection. Examples of strains of vaginal microbiota that can be administered to a subject as set forth herein can include, but are not limited to, Lactobacillus crispatus, Lactobacillus iners, Lactobacillus jensenii, Lactobacillus gasseri, Lactobacillus acidophilus, Lactobacillus rhamnosus, Lactobacillus reuteri, and Bifidobacterium longum. Examples of prebiotics include, but are not limited to, lactitol, inulin, lactulose, and oligosaccharides. Examples of acidifying agents include, but are not limited to lactic acid, acetic acid, ascorbic acid, aspartic acid, citric acid, folic acid, potassium sorbate, and boric acid. In certain embodiments, an acidifying agent is lactic acid. In certain embodiments, at least one bioactive agent may be released at a controlled rate over a predetermined period of time.

In certain embodiments, the one or more vaginal-health-promoting agents can be mixed with the spermicide bio-active agents described above. In certain embodiments, the one or more vaginal-health-promoting agents are separated from the spermicide bio-active agents. For example, the one or more vaginal-health-promoting agents can be stored in reservoirs separated from the reservoirs where the spermicide bio-active agents are stored.

In certain embodiments, a contraceptive device described herein is positioned in the vaginal region of a female reproductive system for a duration of one week to one year (or less than two weeks in emergency contraception embodiments), after which the contraceptive device is removed from the vaginal region of a female reproductive system for a duration of one to three weeks or more weeks. In certain embodiments, a contraceptive device is inserted in the vaginal region of a female reproductive system for a duration of one week, after which the contraceptive device is removed from the vaginal region of the female reproductive system for a duration of one week. In certain embodiments, a contraceptive device is inserted in the vaginal region of a female reproductive system for a duration of two weeks, after which the contraceptive device is removed from the vaginal region of the female reproductive system for a duration of one week. In certain embodiments, a contraceptive device is inserted in the vaginal region of a female reproductive system for a duration of two weeks, after which the contraceptive device is removed from the vaginal region of the female reproductive system for a duration of two weeks. In certain embodiments, a contraceptive device is inserted in the vaginal region of a female reproductive system for a duration of three weeks, after which the contraceptive device is removed from the vaginal region of the female reproductive system for a duration of one to two weeks. In certain embodiments, a contraceptive device is positioned in the vaginal region of a female reproductive system for a duration of month, after which the contraceptive device is removed from the vaginal region of the female reproductive system for a duration of one week to three weeks or more. In certain embodiments, a contraceptive device is installed in a vaginal region of a female reproductive system during ovulation. In certain embodiments, a contraceptive device is installed in a vaginal region of a female reproductive system as needed. In certain embodiments, a contraceptive device is installed in a vaginal region of a female reproductive system for a duration of one year, after which the contraceptive device is removed from the vaginal region of the female reproductive system for a duration of one week to three weeks or more. In certain embodiments, a contraceptive device can be re-used monthly up to one year. For example, a contraceptive device can be inserted in the vaginal region of a female reproductive system for one to three weeks per month, after which the contraceptive device can be removed for one to three weeks and can be re-used the following month for about one to three weeks, or other similar intervals that would be immediately envisaged by the skilled artisan in view of the disclosure contained herein.

A contraceptive device described herein can be used before intercourse for prophylactic contraception. A contraceptive device described herein is as effective as prior art copper IUDs but has much less side effects than the Copper IUDs because of the distance from uterus and duration of use. Specifically, unlike Copper IUDs, a device described herein is not positioned within the uterus and is not placed inside a body for 5 or more years. A contraceptive device described herein also does not have the side effects associated with hormonal approaches because it does not contain hormones.

A contraceptive device described herein can be used, like copper IUDs, within 120 hours of intercourse for emergency contraception. For example, a contraceptive device can be used within about 15 minutes, 30 minutes, 1 hour, 2 hours, 6 hours, 12 hours, 24 hours, 48 hours, 72 hours, 120 hours or any time in between. Due to the ease of insertion and low risk of a foreign body response (like those arising from copper IUDs inserted into the uterus, for example), the embodiments described herein provide advantages compared to prior art devices, which do not provide for accessible emergency contraception using copper ions. For example, and as described further herein, a copper IUD is not easily accessible as an emergency contraceptive device at least because of their insertion requiring a clinical setting and under the supervision of a medical professional, such as a physician. Because a contraceptive device described herein can be positioned inside the vaginal region of a female reproductive system by a subject in need thereof and does not require a clinic setting, it is more accessible and convenient than copper IUDs.

In certain embodiments, one or more vaginal-health-promoting agents can be co-administered with a device described herein. For example, they can be administered prior to, during, and after the use of a device described herein to maintain and restore healthy vaginal environment. In certain embodiments, the one or more vaginal-health-promoting agents are administered upon the removal of a device described herein to restore healthy vaginal environment.

The terminology used herein is for the purpose of describing particular embodiments only and is not intended to be limiting. As used in the Specification and the appended claims, the singular forms “a”, “an” and “the” include plural referents unless the context clearly dictates otherwise.

In the present disclosure, the “female reproductive system” or “uterine reproductive system” can refer to the reproductive system of anyone having the ability to become pregnant. As used herein, the female reproductive system is not limited to individuals identifying as female, rather, it is recognized as a common term for the physiological system supporting fertility and reproduction, including organs such as the uterus, ovaries, fallopian tubes, vagina and cervix.

In the present disclosure, “spermiostatic” or “spermicidal” can mean a substance providing contraceptive benefits by acting to inhibit or reduce the normal cell function of a sperm, for example, by inhibiting a sperm's motility and/or survival, thereby preventing the ability of a sperm to fertilize an egg. As used herein, spermiostatic or spermicidal substances can be used alone or in combination with other contraceptive substances to prevent unwanted pregnancy.

In the present disclosure, a “bioactive agent” can refer to a substance that has an effect on cell functions or behaviors of a living organism. A bioactive agent can be a metal, a chemical compound, a pharmaceutical product (e.g., a drug), and an enzyme.

In the present disclosure, “pearl index” refers to a statistical measure of a pregnancy rate, calculated using the following formula: Pearl Index=(Number of pregnancies×12)×100/(Number of women in the study x Duration of study in months). The lower the pearl index, the lower the pregnancy rate, the better the birth control.

In the present disclosure, the unit “μg/g” is equivalent to “μg/ml” as 1 g of a sample is about 1 ml, “wet weight” refers to weights of samples collected as is, and “dry weight” refers to weights of samples dried before analysis.

In the present disclosure, an “elastomer” or an “elastomeric material” can mean a natural or synthetic polymer having elastic properties, i.e., capable of returning to its original shape after a loading force has been removed.

In the present disclosure, “thermoplastic” can mean any substance having properties of a plastic material when heated (e.g., soft, pliable, moldable etc.), and which harden or solidify when cooled.

In the present disclosure, “molding” can mean securing an object over another object and “overmolding” can mean an injection molding technique for manufacturing a single component comprising two or more materials, such as molding a thermoplastic material over another thermoplastic material.

As used herein, “exposed surface” and “external surface” are interchangeable and “foreign body response” and “foreign body reaction” are interchangeable.

As used herein, “copper”, “copper metal” and “pure copper metal” are interchangeable and can refer to a material containing at least 99.9% of copper.

As used herein, “about” means to encompass variations of ±30%, ±20%, ±10%, ±5%, ±1%, ±0.5%, ±0.1% or any variations that are appropriate to perform as envisaged by a skilled artisan in the field. The numerical ranges referenced herein will be immediately envisaged by the skilled artisan based upon the context in which they are used in the disclosure contained herein.

The following describes example technical solutions of this disclosure with reference to accompanying drawings. Similar reference numerals may have been used in different figures to denote similar components.

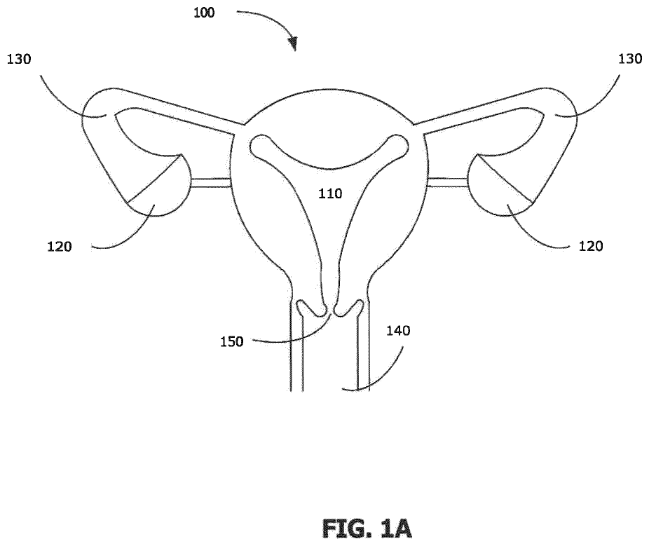

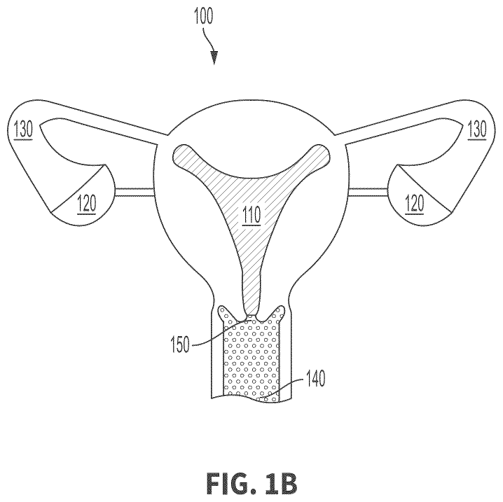

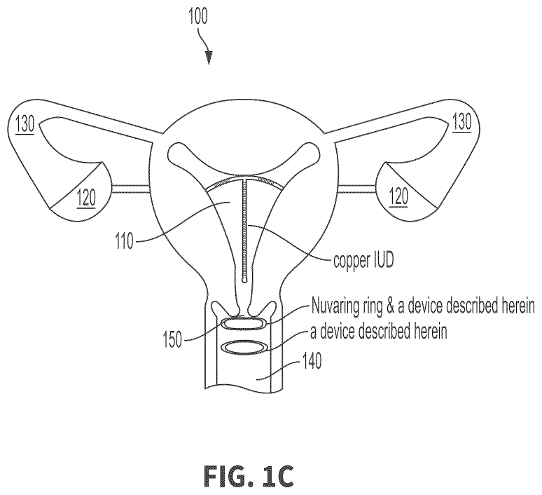

FIG. 1A illustrates an example of a female reproductive system 100 in which the methods and devices described herein can be applied, in accordance with embodiments of the present disclosure. The system 100 has been simplified for ease of understanding and simplistic reference in the embodiments contained herein. The primary internal organs of the female reproductive system 100 include uterus 110, ovaries 120, fallopian tubes 130, vagina (or vaginal tract) 140 and cervix 150. The cervix 150 is the narrow inferior portion of the uterus 110 that projects into the vagina 140. In the process of fertilization, sperms travel along the vaginal tract 140 and enter the uterus 110 through the cervix 150. The cervix produces mucus secretions that become thin and stringy under the influence of high systemic plasma estrogen concentrations, and these secretions can facilitate sperm movement through the reproductive tract. An unfertilized ovum (e.g., egg) stored in one of two ovaries 120 is released into a respective fallopian tube 130 and travels toward the uterus 110. Typically, the ovum may be fertilized by a sperm in the fallopian tube 130 and the fertilized ovum arrives at the uterus where it may implant into the inner wall of the uterus 110 (e.g., endometrium). Following implantation, the fertilized ovum may become an embryo and then develop into a fetus.

FIG. 1B illustrates that, when a cooper IUD is implanted, copper ions are concentrated in the uterus 110 shown as slash shadows and when a device described herein is positioned, copper ions are concentrated in the vaginal and cervical region shown as dotted shadows and in the uterus 110.

FIG. 1C illustrates the positions of a copper IUD, Nuvaring®, and a device describe herein when placed in the female reproductive system. A copper IUD is placed inside the uterus 110, a Nuvaring® ring is placed deep into the cervical region, and a device described herein can be placed in the vaginal region.

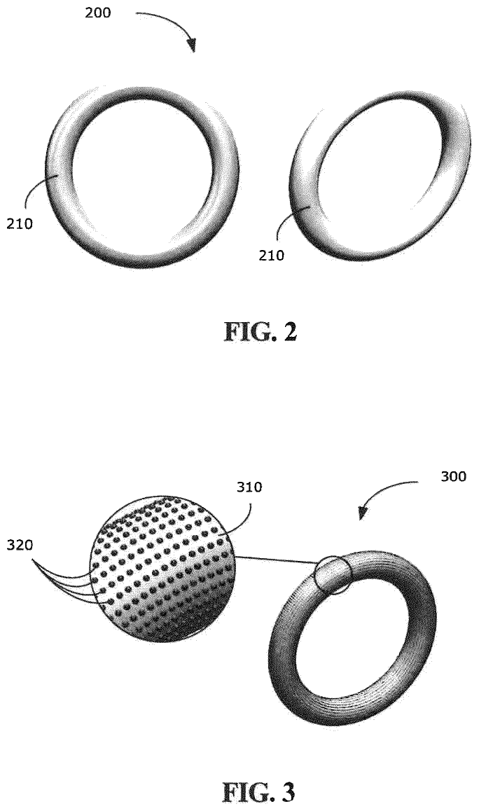

FIG. 2 shows an exemplary contraceptive device in accord with the description contained herein. A contraceptive device is an intravaginal ring 200 comprising a torus-shaped elastomeric body 210.

FIG. 3 shows an exemplary intravaginal ring, in accordance with certain embodiments of the present disclosure. Intravaginal ring 300 has a torus-shaped elastomeric body 310 and plural copper-containing inclusions 320. In certain embodiments, the copper-containing inclusions 320 comprise a bioactive agent comprising copper, such as copper metal or a copper alloy. In certain embodiments, the copper-containing inclusions 320 are dotted on the surface the elastomeric body 310 and dispersed throughout the elastomeric body 310 so that an entire surface of the copper-containing inclusions 320 is exposed. When the intravaginal ring 300 is positioned in the vaginal or cervical region of a female reproductive system 100, the copper-containing inclusions 320 are exposed to the environment in the vaginal region and as a result, copper ions are released into the vaginal region and also into the cervical region and the endometrial region. In certain embodiments, the copper-containing inclusions 320 are embedded within the elastomeric body 310 and dispersed throughout the elastomeric body 310 so that the copper-containing inclusions 320 is partially exposed or is unexposed. When the intravaginal ring 300 is positioned in the vaginal region of a female reproductive system 100, copper ions pass through the elastomeric body 310 and are released into the vaginal region. The size, quantity, and spacing of copper-containing inclusions 320 dotted or embedded within the body 310 are not particularly limited, but can be oriented to optimize the total surface area and positioning of the copper-containing inclusions 320, for controlling a dosage of the copper-containing substance to be optimally released (and therefore confer contraceptive activity) very close to the cervix thereby providing improved contraception compared to prior art devices that are incapable of positioning released copper in this region. In certain embodiments, a total exposed surface area of the copper-containing inclusions 320 is about 50-1000 mm², about 200-600 mm², about 380-600 mm², about 380-400 mm², about 400-500 mm², about 394 mm², about 473 mm², or about 425 mm². The total exposed surface area, unexposed inclusions, and a combination thereof can be adjusted to control release rates of copper ions that can be envisaged by a skilled person in art in view of the disclosure herein.

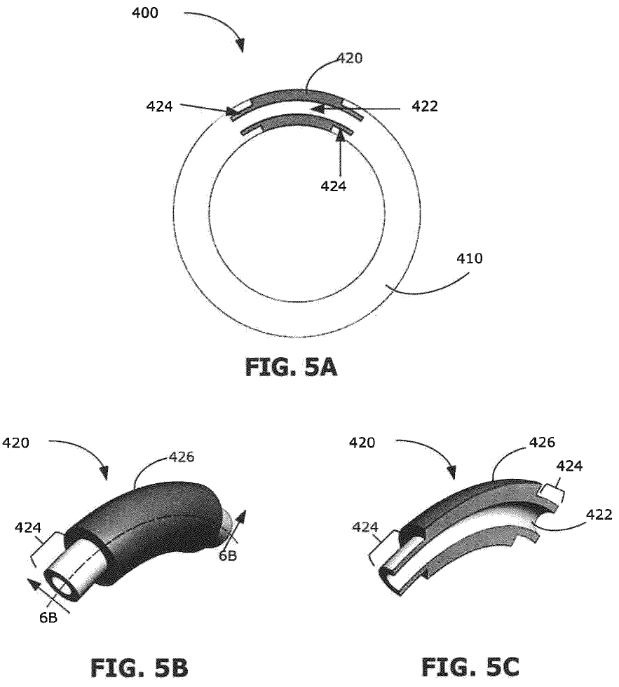

FIGS. 4A-4E show exemplary intravaginal rings, in accordance with certain embodiments of the present disclosure. Intravaginal rings shown in FIGS. 4A-4E (400) have a torus-shaped elastomeric body 410 and one or more copper-containing segments 420. The one or more copper-containing segments 420 are connected or coupled to the elastomeric body 410. The outer surface of one or more copper-containing segments 420 can be in alignment with the outer surface of the elastomeric body 410 (e.g., as described with respect to FIG. 5). When the intravaginal ring 400 is positioned in the vaginal region of a female reproductive system 100, the copper-containing segments 420 are exposed to the environment of the region and as a result, copper ions are released into the vaginal region, as well as the cervical region and the endometrial region through diffusion. The outer surface of one or more copper-containing segments 420 can be coated with a permeable membrane, the outer surface of which is aligned with the outer surface of the elastomeric body 410. The permeable membrane can be used to control release rates of copper ions.

In certain embodiments, the one or more copper-containing segments 420 have a total exterior surface area of about 50-1000 mm². In certain embodiments, the one or more copper-containing segments 420 have a total exterior surface area of about 200-600 mm². In certain embodiments, the one or more copper-containing segments 420 have a total exterior surface area of about 380-600 mm². In certain embodiments, the one or more copper-containing segments 420 have a total exterior surface area of about 380-400 mm². In certain embodiments, the one or more copper-containing segments about 420 have a total exterior surface area of about 400-500 mm². In certain embodiments, the one or more copper-containing segments 420 have a total exterior surface area of about 394 mm². In certain embodiments, the one or more copper-containing segments 420 have a total exterior surface area of about 425 mm². In certain embodiments, the one or more copper-containing segments 420 have a total exterior surface area of about 473 mm². In certain embodiments, the one or more copper-containing segments 420 have an arc of about 40-60 degrees. In certain embodiments, the one or more copper-containing segments 420 have an arc of about 45-55 degrees. In certain embodiments, the one or more copper-containing segments 420 have an arc of about 45-50 degrees. In certain embodiments, the one or more copper-containing segments 420 have an arc of about 45 degrees. In certain embodiments, the one or more copper-containing segments 420 have an arc of about 50 degrees. In certain embodiments, a total exterior surface area of the one or more copper-containing segments and a total exterior surface area of the elastomeric body are present in a ratio between about 1:100 and 1:1, a ratio between about 1:50 and 1:1, a ratio between about 1:20 and 1:1, a ratio between about 1:10 and 1:1, a ratio between about 1:5 and 1:1, and any range in between. In a particular embodiment, the intravaginal ring 400 has a cross-sectional diameter of about 4 mm, an external diameter of about 54 mm, a single copper-containing segment 420 with an arc of about 45 degrees, and an exterior surface area of the single copper-containing segment 420 of about 473 mm². In another particular embodiment, the intravaginal ring 400 has a cross-sectional diameter of about 4.8 mm, an external diameter of about 55 mm, and an exterior surface area of the single copper-containing segment 420 of about 394 mm². In another particular embodiment, the intravaginal ring 400 has a cross-sectional diameter of about 6.35 mm, an external diameter of about 55 mm, a single copper-containing segment 420 with an arc of about 50 degrees, and an exterior surface area of the single copper-containing segment 420 of about 425 mm². The intravaginal ring 400 may comprise one copper-containing segment 420 (as in FIG. 4A), two copper-containing segments 420 (as in FIG. 4B), three copper-containing segments 420, four copper-containing segments 420 (as in FIG. 4C), six copper-containing segments 420 (as in FIG. 4D), 12 copper-containing segments 420 (as in FIG. 4E), or more or any number in between. It is understood that, while each copper-containing segment 420 in FIGS. 4B-4E are shown to be the same size, each copper-containing segment 410 of an intravaginal ring 400 may be different sizes. The size and quantity of copper-containing segments 420 may be determined based on a desired dosage or rate of release of copper ions into the vaginal region, the cervical region, and the endometrial region and/or a desired level of flexibility of the intravaginal ring 400 for ease of insertion and retention of the intravaginal ring in the desired position. In certain embodiments, the copper-containing segments 420 may be arranged in an alternating pattern, for example, alternating with sections of the elastomeric body 410, based on a desired level of flexibility of the intravaginal ring 400 for ease of insertion.

FIG. 5A shows a cross sectional view of the intravaginal ring 400 of FIG. 4A. The intravaginal ring 400 comprises an elastomeric body 410 and one or more copper-containing segments 420. In some embodiments, each copper-containing segment 420 may be an arcuate and tubular-shaped component, having a hollow cavity 422 spanning the length of the copper-containing segment 420, and projections 424 on each end for securing the copper-containing segment 420 to the elastomeric body 410. In some embodiments, the projections 424 can be barbs that have sufficient lengths to grip the inside diameter of the elastomeric body 410 and seal the connection. In some embodiments, the copper-containing segments 420 are copper metal. In certain embodiments, the copper-containing segment 420 of FIG. 5A may be constructed of a rigid or thermoplastic material having properties that enable bonding with an elastomer material. In some embodiments, the copper-containing segment 420 may be constructed of a rigid material, the surface of which can be coated with copper.

In some embodiments, the intravaginal ring 400 may be assembled by inserting the barbs 424 into the elastomeric body 410 where the barbs 424 have sufficient lengths to grip the inside diameter of the elastomeric body 410 and seal the connection. In some embodiments, the intravaginal ring 400 may be assembled by molding (e.g., overmolding), where the elastomeric body 410 is molded directly on to the projections 424, thus coupling the elastomeric body 410 directly to the copper-containing segment 420. In certain embodiments, the projections 424 may extend sufficient lengths to provide for sufficient surface areas for coupling to the elastomeric body 410. In some embodiments, during molding, the hollow cavity 422 may be filled with an elastomeric material to provide mechanical locking of the copper-containing segment 420 with the elastomeric body 410. The copper-containing segment 420 may be secured by other means that would be immediately envisaged by the skilled artisan in the field in view of the disclosure contained herein.

FIG. 5B is a perspective view of the copper-containing segment 420, in accordance with embodiments of the present disclosure. The copper-containing segment 420 comprises a metallic or thermoplastic core, the outer surfaces of which are coated with a copper layer 426. The metallic or thermoplastic core can be between the two projections 424. The thickness of the copper layer 426 may be determined based on a desired dosage or rate of release of copper ions into the vaginal region, the cervical region, and the endometrial region. For example, the copper layer 426 can have a thickness of between about 0.1 mm to 1 cm, between about 0.5 mm to 5 mm, between about 1 mm to 5 mm, between about 1 mm to about 2.5 mm, or any ranges in between. In some embodiments, the metallic or thermoplastic core may be coated with the copper layer 426 first and then coupled with the elastomeric body 410 by inserting protuberances 424 into the elastomeric body 410. In some embodiments, protuberances 424 can include friction-inducing elements, for example, barbs, to help secure and retain the thermoplastic core in the elastomeric layer. Alternatively, the elastomeric body 410 may be molded on the projections 424 of the metallic or thermoplastic core and then the copper layer 426 is coated on the core. The copper coating and the copper layer may be a copper alloy or a pure copper metal, among other copper compositions as described herein.

FIG. 5C is a cut-away view of the copper-containing segment 420 of FIG. 5B, in accordance with embodiments of the present disclosure. The copper-containing segment 420 comprises a hollow cavity 422 extending the length of the copper-containing segment 420, projections 424 extending from each end of the copper-containing segment 420.

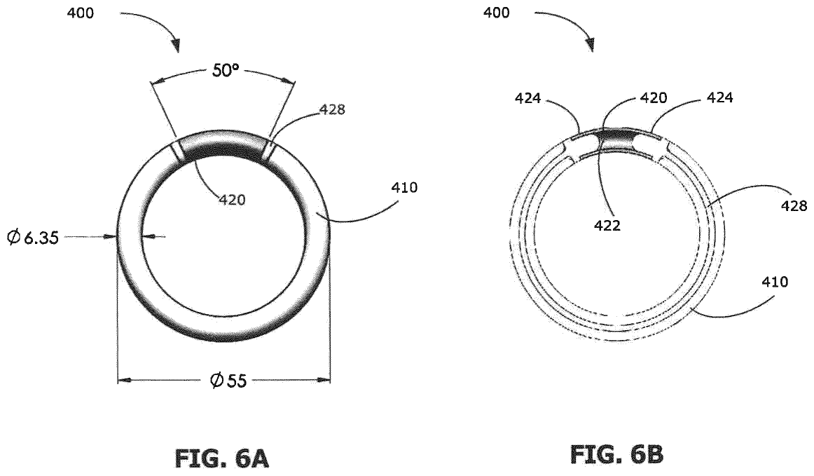

FIG. 6A shows a side view of the intravaginal ring of FIG. 4A, in accordance with embodiments of the present disclosure. The intravaginal ring 400 comprises the elastomeric body 410 and one or more copper-containing segments 420. The one or more copper-containing segments 420 can be coupled to the elastomeric body 410 via a metallic or thermoplastic support ring 428.

In certain embodiments, the one or more copper-containing segments 420 has a total exterior surface area of about 50-1000 mm². In certain embodiments, the one or more copper-containing segments 420 has a total exterior surface area of about 200-600 mm². In certain embodiments, the one or more copper-containing segments 420 has a total exterior surface area of about 380-600 mm². In certain embodiments, the one or more copper-containing segments 420 has a total exterior surface area of about 380-400 mm². In certain embodiments, the one or more copper-containing segments 420 has a total exterior surface area of about 400-500 mm². In certain embodiments, the one or more copper-containing segments 420 has a total exterior surface area of about 394 mm². In certain embodiments, the one or more copper-containing segments 420 has a total exterior surface area of about 425 mm². In certain embodiments, the one or more copper-containing segments 420 has a total exterior surface area of about 473 mm². In certain embodiments, the one or more copper-containing segments 420 has an arc of about 40-60 degrees. In certain embodiments, the one or more copper-containing segments 420 has an arc of about 45-55 degrees. In certain embodiments, the one or more copper-containing segments 420 has an arc of about 45-50 degree. In certain embodiments, the one or more copper-containing segments 420 has an arc of about 45 degrees. In certain embodiments, the one or more copper-containing segments 420 has an arc of about 50 degrees. In a particular embodiment, the intravaginal ring 400 has a cross-sectional diameter of about 4 mm, an external diameter of about 54 mm, a single copper-containing segment 420 with an arc of about 45 degrees, and an exterior surface area of the single copper-containing segment 420 of about 473 mm². In another particular embodiment, the intravaginal ring 400 has a cross-sectional diameter of about 4.8 mm, an external diameter of about 55 mm, and an exterior surface area of the single copper-containing segment 420 of about 394 mm². In another particular embodiment, the intravaginal ring 400 has a cross-sectional diameter of about 6.35 mm, an external diameter of about 55 mm, a single copper-containing segment 420 with an arc of about 50 degrees, and an exterior surface area of the single copper-containing segment 420 of about 425 mm². In still another particular embodiment, the intravaginal ring 400 has a cross-sectional diameter of about 6.35 mm, an external diameter of about 55 mm, two copper-containing segments 420 each having an arc of about 50 degrees, and a total exterior surface area of the two copper-containing segments 420 of about 425 mm². In still another particular embodiment, the intravaginal ring 400 has a cross-sectional diameter of about 4 mm, an external diameter of about 54 mm, two copper-containing segments 420 each having an arc of about 45 degrees, and a total exterior surface area of the two copper-containing segments 420 of about 473 mm². In certain embodiments, a total exterior surface area of the one or more copper-containing segments and a total exterior surface area of the elastomeric body are present in a ratio between about 1:100 and 1:1, a ratio between about 1:50 and 1:1, a ratio between about 1:20 and 1:1, a ratio between about 1:10 and 1:1, a ratio between about 1:5 and 1:1, and any range in between.

FIG. 6B shows a cross-sectional view of the intravaginal ring of FIG. 6A, in accordance with examples of the present disclosure. The intravaginal ring 400 comprises the elastomeric body 410 that is molded onto a support ring 428 and one or more copper-containing segments 420. The support ring 428 can be metallic or thermoplastic. In certain embodiments, the support ring 428 is thermoplastic. In certain embodiments, the support ring 428 has a ring body and two enlarged ends, where the ring body provides support structure over which the elastomeric body can be molded and the two enlarged ends enable the copper-containing segments 420 to couple with the elastomeric body 410. In certain embodiments, the support ring 428 has a ring body and two ends having the same size as the ring body.

In certain embodiments, the elastomeric body 410 is molded over the ring body of the support ring 428 followed by inserting the barbs 424 of the copper-containing segment 420 into the enlarged ends of the support ring 428 to secure the connection between the elastomeric body 410 and the copper-containing 420. In certain embodiments, the copper-containing segment 420 may be mounted on the support ring 428 by inserting each end of the support ring 428 into the hollow cavity 422 of the copper-containing segment 420 prior to the molding of the elastomeric body 410 over the supporting ring 428. In certain embodiments, the copper-containing segment 420 may be mounted on the supporting ring 428 by inserting each end of the support ring 428 into the metallic or thermoplastic core positioned between the two projections 424 prior to the molding of the elastomeric body 410 over the supporting ring 428. The copper-containing segment 420 can be made of copper metal and copper alloys. In certain embodiments, the copper-containing segment 420 is made of copper metal. In certain embodiments, the copper is Cu 101 containing about 99.99% copper. In certain embodiments, the elastomeric body is made of silicone.

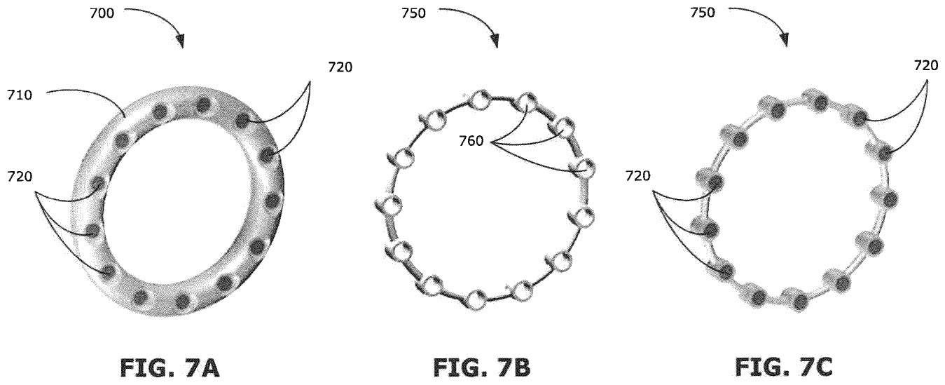

FIG. 7A shows an exemplary embodiment of an intravaginal ring, in accordance with embodiments of the present disclosure. The intravaginal ring 700 has an elastomeric body 710, one or more holes 720 in the elastomeric body 710, and a copper-containing agent within the elastomeric body. The elastomeric body 710 is made of biocompatible polymers. In certain embodiments, the elastomeric body 710 is a silicone elastomer that can be permeable or impermeable. The intravaginal ring 700 is shown in an exemplary embodiment having 13 circular holes 720, however holes 720 may be incorporated into the intravaginal ring 700 can be in other shapes and quantities. For examples, the holes 720 can have a shape of oval, triangle, rhombus, square, rectangle, trapezoid, pentagon, hexagon, or octagon, or any combination thereof. The quantity of the holes 720 can be one, two, three, four, five, six, seven, or more. When the intravaginal ring 700 is positioned in the vaginal region of a female reproductive system 100, the holes 720 allow a copper-containing agent to expose to the fluids of the region and release copper ions into the vaginal region, the cervical region, and the endometrial region.

FIG. 7B shows an exemplary copper-containing agent within the intravaginal ring 700 of FIG. 7A, in accordance with embodiments of the present disclosure. In certain embodiment, a copper-containing agent is a copper wire shown as 750 without apertures 760. A total exterior surface of the copper wire can be between about 50 mm² and 1000 mm², between about 200 mm² and 600 mm², between about 380 mm² and 600 mm², between about 380 mm² and 400 mm², between about 400 mm² and 500 mm², and any ranges in between. In certain embodiment, a thermoplastic skeleton 750 with one or more apertures 760 that fit into the holes 720 as shown in FIG. 7B is used. The one or more apertures 760 can be reservoirs to store a copper-containing agent, which can be copper metal, a copper alloy, a solid copper salt, or a copper-salt solution. In certain embodiments, copper metal, a copper alloy, a solid copper salt or a combination thereof can be filled into the one or more apertures 760 and have exterior surfaces exposed to the environment of the vaginal region when positioned in the region. A total exterior surface of the copper metal, copper alloy, and solid copper salts can be between about 50 mm² and 1000 mm², between about 200 mm² and 600 mm², between about 380 mm² and 600 mm², between about 380 mm² and 400 mm², between about 400 mm² and 500 mm², and any ranges in between. In certain embodiments, a copper-salt solution within a permeable membrane can be stored in the one or more apertures 760. In some embodiments, a dosage resulting from about 50-1000 mm² of exposed copper-containing agent may be used.

In certain embodiments, a copper wire can be placed inside the elastomeric body 710 during its preparation or inserted through the holes 720 after the preparation of the elastomeric body 710. In certain embodiments, the elastomeric body 710 is molded onto the thermoplastic skeleton 750 and a copper-containing agent is filled into the one or more apertures 760.

In some embodiments, a contraceptive device of the present disclosure further comprises a barrier such as a diaphragm device. In certain embodiments, a contraceptive device can be a ring that is not a barrier device like a diaphragm device. In certain embodiments, the contraceptive device described herein is not a barrier device. In certain embodiments described herein, the contraceptive device is not saucer-shaped. In certain embodiments of the contraceptive device described herein, the contraceptive device does not form a barrier over the opening of the cervix. In some embodiments, a barrier may comprise a flexible body that is positioned in the vaginal region of the female reproductive system 100 for providing a physical barrier against sperm migration and fertilization and a bioactive agent comprising copper that is embedded into the flexible body. In some embodiments, a bioactive agent is a copper wire that is coiled along an outer rim of the flexible body, and where a surface of the copper wire is exposed to the environment of the vaginal region, for releasing copper ions for hindering sperm migration and fertilization. A barrier provides an added benefit of combining a physical barrier for blocking sperm from passing through the cervix, with the spermicidal effect of copper for highly effective contraception.

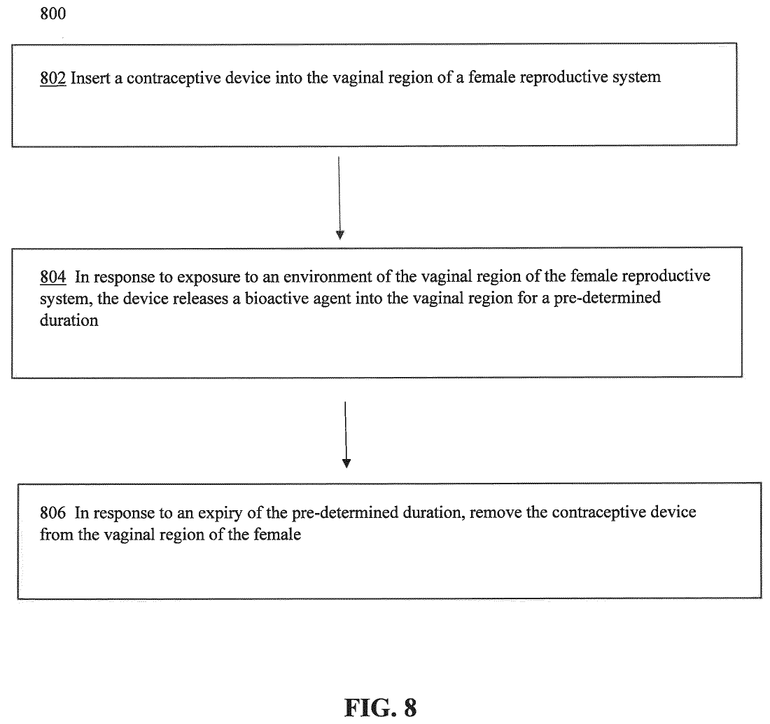

FIG. 8 illustrates a method of using a contraceptive device described herein. The method 800 begins at step 802, where the contraceptive device is inserted into the vaginal region of the female reproductive system 100 by a subject in need thereof and/or with assistance from a device applicator. In certain embodiments, a contraceptive device described herein can easily inserted by a subject in need thereof, rather than by a clinical professional or in a clinical setting, unlike prior art copper IUDs that require administration in a clinical setting, which can be painful and inapplicable for use as an emergency contraceptive. A contraceptive device described herein may be used for a planned contraceptive and/or for an emergency contraceptive. A contraceptive device described herein may be inserted into the vaginal region prior to intercourse or within 120 hours of intercourse and may remain installed for a duration of two weeks, three weeks, or one year, or any duration in between.

At step 804, in response to exposure to an environment of the vaginal region of the female reproductive system 100, a bioactive agent containing copper may be released at a particular rate into the vaginal region, the cervical region, and the endometrial region to elicit a spermicidal or spermiostatic effect on sperm that may be present in the vaginal and/or the cervical region. In certain embodiments, a bioactive agent containing copper is released at a controlled rate and/or at a controlled dosage. In certain embodiments, a contraceptive device described herein is configured to continually release the bioactive agent containing copper for the pre-determined duration, for example, providing a reliable method of contraception for the entirety of the pre-determined duration. In certain embodiments, the pre-determined duration is one day to four weeks or more.

In certain embodiments, the copper concentrations in the uterus are about 3.0-20 μg/g or about 3.0-20.0 μg/ml (dry weight) from day 3 to year 5 following the insertion of a device described herein. For example, the copper concentration in the uterus (dry weight) can be about 3.9-4.2 μg/g, 3.5 μg/g, 3.9 μg/g, 4.0 μg/g, 4.1 μg/g, 4.2 μg/g, 4.3 μg/g, 4.4 μg/g, 4.5 μg/g, 5.0 μg/g, 5.5 μg/g, 6.0 μg/g, 6.5 μg/g, 7.0 μg/g, 7.5 μg/g, 8.0 μg/g, 8.5 μg/g, 9.0 μg/g, 9.5 μg/g, 10.0 μg/g, 10.5 μg/g, 11.0 μg/g, 11.5 μg/g, 12.0 μg/g, 12.5 μg/g, 13.0 μg/g, 13.5 μg/g, 14.0 μg/g, 14.5 μg/g, 15.0 μg/g, 15.5 μg/g, 16.0 μg/g, 16.5 μg/g, 17.0 μg/g, 17.5 μg/g, 18.0 μg/g, 18.5 μg/g, 19.0 μg/g, 19.5 μg/g, 20.0 μg/g, or any amount in between. In certain embodiments, the copper concentrations in the uterus (dry weight) on day 3 following the insertion of a device described herein are about 3.9-10.0 μg/g. For example, the copper concentrations in the uterus on day 3 following the insertion of a device described herein can be about 3.9-4.2 μg/g, 3.9 μg/g, 4.0 μg/g, 4.1 μg/g, 4.2 μg/g, 4.3 μg/g, 4.4 μg/g, 4.5 μg/g, 5.0 μg/g, 5.5 μg/g, 6.0 μg/g, 6.5 μg/g, 7.0 μg/g, 7.5 μg/g, 8.0 μg/g, 8.5 μg/g, 9.0 μg/g, 9.5 μg/g, 10.0 μg/g, or any amount in between.

In certain embodiments, the copper concentrations in the uterus can be about 0.5-5.0 μg/g (dry weight) or about 0.5-5.0 μg/ml (wet weight) from day 3 until removal of the device (e.g., one month, two months, three months, six months, one year, two years, three years, four years, or up to about five years or more) following the insertion of a device described herein. The copper concentration in the uterus (wet weight) can be about 0.8-1.8 μg/g, 0.9-1.8 μg/g, 10.5 μg/g, 0.6 μg/g, 0.7 μg/g, 0.8 μg/g, 0.9 μg/g, 1.0 μg/g, 1.01 μg/g, 1.2 μg/g, 1.3 μg/g, 1.04 μg/g, 1.5 μg/g, 1.6 μg/g, 1.7 μg/g, 1.8 μg/g, 1.9 μg/g, 2.0 μg/g, 2.5 μg/g, 3.0 μg/g, 3.5 μg/g, 4.0 μg/g, 4.5 μg/g, 5.0 μg/g, or any amount in between. In certain embodiments, the copper concentrations in the uterus on day 3 following the insertion of a device described herein are about 0.9-3.0 μg/g (wet weight). For example, the copper concentrations in the uterus on day 3 following the insertion of a device described herein can be about 0.8-1.8 μg/g, 0.9-1.8 μg/g, 0.9 μg/g, 1.0 μg/g, 1.1 μg/g, 1.2 μg/g, 1.3 μg/g, 1.4 μg/g, 1.5 μg/g, 1.6 μg/g, 1.7 μg/g, 1.8 μg/g, 1.9 μg/g, 2.0 μg/g, 2.5 μg/g, 3.0 μg/g, or any amount in between.

It is understood that the copper concentration in the uterus will increase after inserting a copper-containing device described herein, but that the copper concentration may reach an equilibrium amount as set forth herein that maintains an effective copper concentration in the cervical region, vaginal region, and/or uterus during the period of time that a contraceptive device described herein remains in a subject's vaginal region. In certain embodiments, the copper concentration in the uterus increases for about a week, two weeks, three weeks, six months, one year, two years, three years, or more following inserting a device described herein.

In certain embodiments, a device described herein has a copper release for about one week to five years. For example, a device described herein can have a copper release for about one week, two weeks, three weeks, one month, two months, six months, one year, two years, three years, four years, five years, or any time in between.

In certain embodiments, a device described herein can have a pearl index of about 0.8-5, about 0.8-1.5, about 0.9-1.4, about 0.8-1.3, about 1.0-1.5, about 0.9-1.1, about 0.8-1.0, about 1.0-3.5, about 0.9-4.5, about 1.8-5.0, or about 1.1-1.2. For example, a device described herein can have a pearl index of about 0.8, 0.9, 1.0, 1.1, 1.2, 1.3, 1.4, 1.5, 2, 3, 4, 5, or any value in between.

At step 806, in response to reaching the pre-determined duration or alternatively, in response to the user determining that use of the contraceptive device is no longer desired, the contraceptive device may be removed from the vaginal region of the female reproductive system by the subject in need thereof. In some embodiments, the contraceptive device may be removed from the vaginal region with assistance from a device applicator or removal tool. In certain embodiments, the contraceptive device is easily removed by the subject in need thereof, rather than by a clinical professional or in a clinical setting.

Although the present disclosure describes methods and processes with steps in a certain order, one or more steps of the methods and processes may be omitted or altered as appropriate. One or more steps may take place in an order other than that in which they are described, as appropriate.

The present disclosure may be embodied in other specific forms without departing from the subject matter of the claims. The described example embodiments are to be considered in all respects as being only illustrative and not restrictive. Selected features from one or more of the above-described embodiments may be combined to create alternative embodiments not explicitly described, features suitable for such combinations being understood within the scope of this disclosure.

All values and sub-ranges within disclosed ranges are also disclosed. Also, although the systems, devices and processes disclosed and shown herein may comprise a specific number of elements/components, the systems, devices and assemblies could be modified to include additional or fewer of such elements/components. For example, although any of the elements/components disclosed may be referenced as being singular, the embodiments disclosed herein could be modified to include a plurality of such elements/components. The subject matter described herein intends to cover and embrace all suitable changes in technology.

### EXAMPLES

### Example 1: Animal Study to Assess the Efficacy of a Contraceptive Device

A contraceptive device illustrated in FIG. 4A was studied for its efficacy in animals by determining copper concentrations in the vaginal fluids and the uterus. The study was conducted by Charles River Laboratories. The device has an outer diameter of about 55 mm and a cross-sectional diameter of about 4.8 mm ( 3/16″). The copper segment in the device has an exposed surface area of about 394 mm². The device was boiled at 100° C. (+/−1° C.) in deionized water for approximately 5 minutes and sealed in sterile bags afterwards prior to the study. Female sheep (S Ovis aries species, Dorset×Rideau Arcott Hybrids strain, supplied by Bakerstone) were used due to their physiological and anatomical similarities to human reproductive systems. Two young adult sheep (2-4.5 years old) and having weights between 70-75 kg were selected for the study.

Prior to the study, both sheep had environmental acclimation for 21 days, during which the sheep were housed together in one room with concrete floors covered by chip bedding and fed with pellets (ENVIGO Teklad Ruminant Food or equivalent) and tap water. To induce estrus and ovulation, the sheep were intramuscularly injected with Estrumate (prostaglandin; 1 mL) eleven days before the study (Day −11) and then with Estrumate (prostaglandin, 1 mL) and Novormon (equine chorionic gonadotrophin, 2 mL) one day before the study (Day −1). Between Day −11 and Day −1, the sheep were habituated to be handled in a sling for at least 3 sessions during the estrus synchronization period.

On the day of the start of the study (Day 0), one contractive device was inserted into each sheep and securely placed in a horizontal position as high as possible in the cranial vagina to prevent slipping out. Prior to the insertion, vaginal fluids and vaginal swabs were collected as baseline vaginal samples for vaginal copper concentration assessment. Vaginal fluids were collected in sterile tubes by touching the walls of the vagina with the sterile tubes. Vaginal swabs were collected by gently sliding swabs high into the cranial vagina, touching the walls of the vagina with the heads of the swabs, and gently rotating the swabs at least 5 times in a clockwise direction to absorb as much moisture as possible. On Day 1 (between 24 hours and 48 hours following the insertion), Day 2 (between 48 hours and 72 hours following the insertion), and Day 3 (between 72 hours and 80 hours following the insertion), vaginal fluids and swabs were collected from both sheep each day using the above methods. The tubes and the swabs were weighed before and after the collection to determine the net weights of the collected vaginal fluids and vaginal swabs. The collected vaginal fluids and swabs were snap frozen on dry ice and stored at −80° C. until the copper concentration analysis.

On Day 3, an endometrial biopsy was performed on one sheep using a laparoscopy. Under anesthesia, an incision was made at the base of the right uterine horn using a Halstead hemostat, and through it a sterile flocked-headed swab was introduced to collect endometrial secretions. The swab was placed in a tube after the collection. Both the tube and swab were weighed before and after the collection to determine the net weight of the collected endometrial swab sample. The endometrial sample was snap frozen on dry ice and then stored in a −80° C. freezer. After removal of the swab, pipelle endometrial biopsy was conducted to collect endometrial secretions from the right and left uterine horns. Every sample was placed in a separate and appropriately identified sterile tube and placed on dry ice and stored in a −80° C. freezer.

Copper concentrations in the vaginal fluids, vaginal swabs, and endometrial_samples were measured using inductively coupled plasma mass spectrometry (ICPMS). For the pipelle endometrial biopsy samples, copper concentrations were first measured based on the wet weights of the samples (using the collected samples as is) and then measured based on the dry weight of the samples (drying the collected samples prior to analysis). Each sample was measured three times and copper concentrations were reported as mean±standard deviation. The results are showed in Tables 1-3 below.

Table 1 shows that the copper concentrations in the vaginal swabs and vaginal fluids increased significantly each day from day 0 to day 3 and on day 3, the vagina had a very high concentration of copper. Table 2 shows that on day 3, the right horn of the uterus had the copper concentrations of about 0.9 μg/g (wet weight) and 4.2 μg/g (dry weight) and the left horn of the uterus had the copper concentrations of about 1.04 μg/g (wet weight) and 3.9 μg/g (dry weight). Table 3 shows that on day 3, the right horn of the uterus had the copper concentrations of about 0.89 μg/g (wet weight) and the left horn of the uterus had the copper concentrations of about 1.74 μg/g (wet weight). It was reported that a copper IUD having a total copper surface area of 175 mm² resulted in the copper concentration of about 0.67 μg/g (wet weight) in the human uterus between month one and month six following the IUD insertion. It was also reported that (i) this copper IUD has a contraception efficacy of 92.2%, (ii) another copper IUD having the same total copper surface area has a Peral Index of 0.96, (iii) NOVA-T® (i.e., a copper IUD) having a total copper surface area of 200 mm² shows a pregnancy rate of 1.26/100 patient-years (i.e., also effective in contraception), and (iv) a copper concentration of 3.9 μg/g (dry weight) or lower in the uterus can be effective for contraception. The contraceptive device described herein yields uterine copper concentrations of at least about 0.90 μg/g (wet weight, thereby exceeding a 0.67 μg/g wet weight threshold which has been deemed to be an effective copper concentration, which arises from prior art copper IUDs) and 3.9 μg/g (dry weight) on day 3, indicating that the contraceptive device described herein is highly effective for contraception both as a preventative device and for emergency contraception. Given the copper concentration achieved on day 3 in the uterus and vagina, the contraceptive device described herein can be used for emergency contraception and is more effective than the copper IUDs reported because it takes the copper IUDs much longer (i.e., at least one month) to release such a high concentration of copper and this high copper concentration is only in the uterus, not in the vagina.

TABLE 1
Copper Concentrations in Sheep Vaginal Fluids

| | Day 0 | Day 1 | Day 2 | Day 3 |
|---|---|---|---|---|
| Copper concentration in the | 0.041 ± 0.0003 | 0.539 ± 0.013 | 1.03 ± 0.01 | 10.62 ± 0.09 |
| vaginal swab (μg/swab) | | | | |
| Copper concentration (μg/g of | 1.60 ± 0.01 | —* | 57.5 ± 0.6 | 136 ± 1 |
| vaginal fluid) | | | | |

*Copper concentration on Day 1 was not listed because the sample failed QC test.

TABLE 2
Copper Concentrations in Sheep Pipelle
Endometrial Biopsy Samples on Day 3

| | Dry | Wet | Dry | Wet | |
|---|---|---|---|---|---|
| | weight | weight | weight | weight | Moisture |
| | (μg/g) | (μg/g) | (g) | (g) | (%)* |
| left horn | 3.9 ± 0.1 | 1.04 ± 0.03 | 0.0176 | 0.0654 | 73.1 |
| right horn | 4.2 ± 0.1 | 0.90 ± 0.03 | 0.0139 | 0.0651 | 78.6 |

*Moisture (%) refers to moisture contents in wet samples.
Dry samples did not have moisture contents.

TABLE 3
Copper Concentrations in Sheep Endometrial
Biopsy Swab Samples on Day 3

| | Cu concentration | Cu mass | Uterine |
|---|---|---|---|
| | (μg/g) | (μg/swab) | fluid (g) |
| right horn | 0.89 ± 0.03 | 43 ± 1 | 0.0483 |
| left horn | 1.74 ± 0.07 | 76 ± 3 | 0.0435 |

### Example 2: Human Study to Assess the Efficacy of a Contraceptive Device

The efficacy of the contraceptive device used in Example 1 was evaluated in human by determining copper concentrations in the vaginal fluids and the uterus.

On the day of the start of the study, the contractive device was inserted into the vagina of a biological female. On each day of day 3 and day 3 after the device insertion, a vaginal swab was collected by inserting the soft tip end of a sterile swab (from STI kit test) into the vagina about 2 inches (5 cm) past the opening of the vagina and rotating the swab for 10 to 30 seconds to ensure the swab touched the walls of the vagina so that moisture was absorbed by the swab. On day 3 after the device insertion, an endometrial biopsy was conducted by a licensed gynecologist in the standard manner to collect an endometrial biopsy sample. Copper concentrations in the vaginal swabs and the uterus were analyzed using ICPMS. The results are shown in Table 4.

The results show that (i) copper concentrations in vaginal increased from day 3 to day 5, which is consistent with the results from the sheep vagina and (ii) on day 5, the uterus had copper concentrations of about 1.12 μg/g (wet weight) and 4 μg/g (dry weight), similar to the copper concentrations in the sheep uterus on day 3 in Example 1. The results further demonstrate the effectiveness of a device described herein for emergency contraception because of the copper concentrations on day 3 and can be used for prophylactic contraception because of the long-lasting release.

TABLE 4
Copper Concentrations in Human Vaginal Swabs and Endometrial Biopsy Samples

| | Copper concentration | | |
|---|---|---|---|
| | in the Vaginal Swab | Copper concentration in | Copper concentration in the |
| | (μg/swab) | the Vaginal Swab (μg/ml) | Endometrial Biopsy (μg/g) |
| Day 3 | 3.04 ± 0.04 | 152 ± 2 | 1.12 ± 0.02 (wet weight; |
| | | | having a moisture content of |
| | | | 72%); 4.02 ± 0.06 (dry weight) |
| Day 5 | 3.47 ± 0.14 | 174 ± 7 | — |

### Example 3: Animal Study to Assess the Safety Tolerability and Efficacy of a Contraceptive Device

A contraceptive device illustrated in FIG. 4A or FIG. 4B will be studied for its safety, tolerability, and efficacy in animals. Sheep of 1-4.5 years of age with a weight of 45-100 kg and having previous pregnancies of 0-2 times will be obtained in January of the study year. The study will be conducted from March to June since these are typical breeding months. The sheep will be freely fed with chow and water at equal frequencies and housed indoors on 12/12 light/dark cycle for six weeks prior to the study and throughout the study duration. A total of 20 sheep will be randomly divided into two groups, where group A will be received a contraceptive device and group B is the control group. Vaginal dimensions of each sheep will be measured to determine the size of a contraceptive device to be inserted.

At day 1 of the study, 5 sheep from Group A sheep will be anesthetized and a contraceptive device will be inserted into each of their vaginal regions. At day 2, all the sheep will be induced for estrus either (i) by treatment of progesterone followed by exposing them to a ram after minimum 6 weeks apart from them or (ii) by treatment with progesterone followed by pregnant mare serum gonadotropin. At day 5 of the study, frozen ram semen will be thawed in a 37° C. water bath for 30 seconds, after which, motility and morphology analysis of sperms will be conducted under a microscope to ensure the ram semen is adequate for use. 60 hours after inducing estrus, all the sheep will be artificially inseminated with the ram semen. At day 35, all the sheep will be assessed for pregnancy. At day 36, the non-pregnant sheep will be induced for estrus and 60 hours after, will be artificially inseminated with the ram semen again.

The efficacy of a contraceptive device described herein for emergency contraception will also be evaluated. 24 hours after insemination, another five sheep from Group A will be anesthetized and inserted a contraceptive device. 72 hours post insemination, still another five sheep from Group A will be anesthetized and inserted a contraceptive device. 120 hours after insemination, the last five sheep from Group A will be anesthetized and inserted the contraceptive device.

At day 71, all the sheep will be assessed for pregnancy using serum testing. From day 1 to day 71, vaginal fluids will be collected to determine the copper concentrations. During the study, the sheep from Group A will be monitored for signs of adverse effects such as infection and irritation via weekly veterinary vaginal exams.

### Example 4: The Safety, Tolerability, and Efficacy of a Contraceptive Device in Human

A contraceptive device described herein and illustrated in either FIG. 4A or FIG. 4B will be assessed in a pivotal clinical trial for its safety, tolerability, and efficacy.

A total of 200 individuals between the ages of 18 and 40 will participate in the study. All the participants have potential to become pregnant, good general health status, regular menstrual cycles between 24 and 32 days, willingness to become pregnant if device fails, willingness to stop using any current contraception, willingness to have regular sex throughout their cycles and ability to give informed consent. The study will be conducted in accordance with the ethical principles outlined in the Tri-Council Policy Statement: Ethical Conduct for Research Involving Humans (TCPS 2).

Each participant will insert a contraceptive device into the vagina and leave in place for three weeks of the menstrual cycle, with removal on day 26 of the menstrual cycle until day five of the following menstrual cycle. Data will be collected at various time points throughout the study period. Prior to study initiation, baseline demographic and medical information will be collected from each participant. During the study, participants will be scheduled for regular follow-up visits at two weeks, one month, two months, three months, five months and seven months, which will include clinical assessments, laboratory tests, and completion of questionnaires to evaluate pregnancy rates, safety, tolerability, and any adverse events. The specific data collected will include serum copper and pregnancy testing, swabs to check vaginal pH, vaginal copper concentration and general vaginal tissue health, survey answers about ease of insertion, comfort, irritation, partner comments if applicable, ease of removal and any concerns of use during sex. Descriptive statistics will be used to summarize demographic characteristics and baseline data. Safety and tolerability will be assessed by examining the incidence and severity of adverse events reported by the participants. Pregnancy rates will be documented and analyzed appropriately.

### Example 5: Efficacy for Emergency Contraception in Human

A contraceptive device described herein and illustrated in either FIG. 4A or FIG. 4B will be assessed for the efficacy in emergency contraception as compared to a copper IUD.

Individuals between the ages of 18 and 40 will participate in the study. The participants will be divided into two groups. One group will insert a contraceptive device described herein and the other group will insert the copper IUD into the vagina within 5 days after intercourse. Following the insertions, copper concentrations in the subject's vagina and uterus will be determined in both groups from day 1 to day 5. Data including pregnancy testing, swabs to check vaginal pH will also be collected and analyzed.

1. A contraceptive device configured for insertion into the vagina of a female reproductive system of a subject, the contraceptive device comprising:
a torus-shaped elastomeric body; and
at least one bioactive agent having spermicidal or spermiostatic properties coupled to the elastomeric body,
wherein the contraceptive device is configured to release an effective amount of the at least one bioactive agent into the vaginal region, cervical region, and endometrial region of the female reproductive system when the contraceptive device is positioned into the vaginal region of the female reproductive system.

2. The contraceptive device of claim 1, wherein the at least bioactive agent is a copper-containing agent.

3. The contraceptive device of claim 2, wherein the copper-containing agent is copper, a copper alloy, a copper salt, or a combination thereof.

4. The contraceptive device of claim 3, wherein the copper-containing agent is copper.

5. The contraceptive device of claim 4, wherein the elastomeric body is dotted with the copper as a plurality of inclusions and the surface of the copper is in contact with the environment of the vaginal region of the female reproductive system.

6. The contraceptive device of claim 1, wherein the at least one bioactive agent further comprises one or more vaginal-health-promoting agents.

7. The contraceptive device of claim 6, wherein the one or more vaginal-health-promoting agents are selected from the group consisting of acidifying agents, vaginal microbiota, prebiotics, or a combination thereof.

8. The contraceptive device of claim 7, wherein the acidifying agent is lactic acid.

9. The contraceptive device of claim 7, wherein the vaginal microbiota are Lactobacillus Lactobacillus crispatus, Lactobacillus iners, Lactobacillus jensenii, Lactobacillus gasseri, Lactobacillus acidophilus, Lactobacillus rhamnosus, Lactobacillus reuteri, Bifidobacterium longum, or a combination thereof.

10. A method for emergency contraception comprising:
inserting an emergency contraceptive device into the vaginal region of a female reproductive system after intercourse, wherein the emergency contraceptive device comprises a torus-shaped elastomeric body and at least one bioactive agent having spermicidal or spermiostatic properties coupled to the elastomeric body.

11. The method of claim 10, wherein the at least bioactive agent is a copper-containing agent.

12. The method of claim 11, wherein the copper-containing agent is copper, a copper alloy, a copper salt, or a combination thereof.

13. The method of claim 12, wherein the copper-containing agent is copper.

14. The method of claim 13, wherein the copper is coupled to the elastomeric body as one or more segments.

15. The method of claim 14, wherein the elastomeric body has a cross sectional diameter of about 3.0-10 mm and an external diameter of about 30-90 mm.

16. The method of claim 14, wherein the one or more copper segments have an arc of about 40-60 degrees.

17. The method of claim 14, wherein the one or more copper segments have a total exterior surface area of about 50-1000 mm².

18. The method of claim 10, wherein the emergency contraceptive device is co-administered with one or more vaginal-health-promoting agents.

19. The method of claim 18, wherein the one or more vaginal-health-promoting agents are selected from the group consisting of acidifying agents, vaginal microbiota, prebiotics, or a combination thereof.

20. The method of claim 19, wherein the acidifying agent is lactic acid.

21. The method of claim 19, wherein the vaginal microbiota are Lactobacillus Lactobacillus crispatus, Lactobacillus iners, Lactobacillus jensenii, Lactobacillus gasseri, Lactobacillus acidophilus, Lactobacillus rhamnosus, Lactobacillus reuteri, Bifidobacterium longum, or a combination thereof.

22. The method of claim 10, wherein the at least one bioactive agent further comprises one or more vaginal-health-promoting agents.

23. The method of claim 10, wherein the emergency contraceptive device is positioned in the vaginal region of a female reproductive system within 120 hours of intercourse and for a duration of less than two weeks, after which the contraceptive device is removed from the vaginal region of a female reproductive system for a duration of one to three weeks or more weeks.

24. The method of claim 10, wherein the emergency contraceptive device is positioned in the vaginal region of a female reproductive system within 120 hours of intercourse and for a duration of from one week to a year, after which the contraceptive device is removed from the vaginal region of a female reproductive system for a duration of one to three weeks.

25. The method of claim 10, wherein the emergency contraceptive device is positioned in the vaginal region of a female reproductive system within 48 hours of intercourse for emergency contraception.

26. The method of claim 10, wherein the emergency contraceptive device is positioned in the vaginal region of a female reproductive system within 24 hours of intercourse for emergency contraception.

27. A method for planned contraception comprising:
inserting a contraceptive device into the vaginal region of a female reproductive system prior to intercourse, wherein the contraceptive device comprises a torus-shaped elastomeric body and at least one bioactive agent having spermicidal or spermiostatic properties coupled to the elastomeric body.

28. The method of claim 27, wherein the contraceptive device is positioned in the vaginal region of a female reproductive system prior to intercourse and for a duration of between one week and one year, after which the contraceptive device is removed from the vaginal region of a female reproductive system for a duration of one to three weeks or more weeks.

29. A method for making a contraceptive device comprising:
providing a thermoplastic support ring;
molding an elastomeric material over the thermoplastic support ring to form a torus-shaped elastomeric body having two ends;
providing an arcuate and tubular-shaped copper metal containing two barbs; and
inserting the barbs into each end of the elastomeric body.
